(19) United States
(12) Patent Application Publication
(10) Pub. No.: US 2007/0141074 A1
(43) Pub. Date: Jun. 21, 2007

(54) Agents for the inhibition of virus replication through regulation of protein folding

(75) Inventor: Ulrich Schubert, Jena (DE)

Correspondence Address: JORDAN AND HAMBURG LLP, 122 EAST 42ND STREET, SUITE 4000, NEW YORK, NY, 10168, US

(73) Assignee: ViroLogik GmbH, Erlangen (DE)

(21) Appl. No.: 10/584,934

(22) PCT Filed: Dec. 30, 2004
(86) PCT No.: PCT/EP04/53739

§ 371 Date: Jun. 30, 2006

(30) Foreign Application Priority Data
Dec. 31, 2003 (DE) 103 61 944.5

(51) Int. Cl. A61K 39/12

(52) U.S. Cl. 424/186.1; 514/12

(57) ABSTRACT

The invention concerns agents for the treatment of acute and chronic infections with human and animal pathogenic viruses which assemble along the cell membrane and are released through budding on the surface of the cell. Hereunto count especially causative agents of infectious diseases such as AIDS, hepatitis, hemorrhagic fever, SARS, smallpox, measles, polio or the flu. The subjects of the invention are agents that contain inhibitors of the protein folding as active components. Hereunto count inhibitors of cellular folding enzymes (the enzymatic chaperones) as well as substances that disturb the folding of proteins through chemical chaperones. The following substance classes and their derivates belong thereunto: Geldanamycin, Deoxyspergualin, 4-PBA or Herbimycin A. Due to these agents the highly organised processes of the assembly and the proteolytical maturation of virus structure proteins is disturbed. As a result the release and production of infectious decendent viruses is prevented.

### BACKGROUND OF THE INVENTION

The invention concerns agents for the treatment of acute and chronic infections with both for human and animal pathogenic viruses that assemble at the cell membrane and are released from the cell's surface through budding. Mainly belonging into this field are viruses causing infectious diseases such as AIDS, hepatitis, hemorrhagic fever, SARS, smallpox, measles, polio and flu. The main object of the invention are substances that contain inhibitors of the protein folding as active ingredients. These include inhibitors of cellular folding enzymes (the enzymatic chaperones) and substances which deregulate the protein folding through chemical chaperones. Due to the application of these agents the highly organised process of assembly and of proteolytical maturation of virus structure proteins is disturbed. As a cause of this, the release and production of infectious progeny viruses is blocked. These agents contain a wide spectrum of efficacy and can therefore be used as new broadband virostatica for the prevention or as a therapy for treatment of diverse virus infections.

The so-called processes of virus replication include the denovo synthesis of virus proteins, in which normally, after the activation of the viral genetic expression, first the virus structure proteins are expressed. These structure proteins are then integrated into the process of assembly and of formation of viral sub-structures. With enveloped viruses this process generally occurs on cellular membranes, mostly at the inner side or the plasma membrane. Alternatively, there is also the chance that first virus proteins assemble to virus-like particles in the cytosol or the nucleus at first and later these virus-like particles are enveloped with a lipid membrane during the budding processes at the cell membrane. This leads to the formation of a virus bud, which is actively produced to the outside of the cell membrane and is finally detached from the cell membrane as a progeny virion.

The principals of the later processes of virus replication will be described using HIV as an example. The main components of the HIV structure proteins are translated as three polyproteins: Gag and Gag-Pol for the inner core proteins and viral enzymes, and Env for the viral surface proteins. Membrane targeting signals located in the NH₂-terminal domain of Gag are relevant for the transport of Gag and the insertion into the cell membrane. In the case of HIV-1, the complete proteolytic maturation of the Gag polyprotein Pr55 results in the formation of the Matrix (MA), the Capsid (CA) along with the Nuclaocapsids (NC) and the COOH-terminaled p6^{gag} proteins. HIV-virions are generally released from the plasma membrane as immature, non-infectious virus particles; this process is known as virus budding. Immediately after or in concert with virus budding the proteolytical processing of Gag and Gag-Pol-polyproteins is initiated by activation of the viral protease (PR). The proteolytical maturation of released virions subsequently results in morphological alterations. Generally, the condensation of the inner core results in the formation of a cone-shaped core cylinder that is typical for mature virus particles (summarized in Kräusslich and Welker, 1996; Swanstrom and Wills, 1997).

Small molecule drugs, for example Glycerol, Trimethylamins (such as Trimethylamin-N-oxid (TMAO)), various amino acid derivates (such as Betain) and also deuterized water (D₂O) were described as “chemical chaperones”. They are known for modulating the protein folding through the regulation of the amount of water bond to the protein structure (Permutter, 2002; Diamant et al., 2001; Gekko & Timasheff, 1981).

It has been described that Geldanamycin interacts with the chaperone Hsp90 and that through this the folding of proteins, especially after a heat shock, is regulated. Geldanamycin hinders the dissociation of Hsp90 from the substrate in particular, and thus causing its inactivation (Whitesell et al., 1994; Schneider et al. 1996).

Deoxyspergualin (dsg, a α-hydroxyglycyl, 7-guanidinoheptanoyl peptidomimetic) is a synthetic analogue to the naturally occurring spergualin, which was isolated from Bacillus laterosporus and shows potent immunosuppressive effects (Takeuchi et al., 1981; Nemoto et al., 1987; Tepper et al., 1991; Dickneite et al., 1987). DSG interacts not only with the proteins of the Hsp70 and Hsp90 families, but also with the constitutively expressed proteins of the Hsc70 family (Nadler et al., 1992).

Further inhibitors of molecular chaperones are Sodium-4-phenylbutyrate (4-PBA), which blocks Hsc70, and Herbimycin A, which blocks Hsp90.

### SUMMARY OF THE INVENTION

The main function of the invention is to create agents that can be used for the treatment of acute and chronic infectious diseases caused by human and animal pathogenic viruses. These viruses are known to assemble inside the cell, preferably at the cell membrane and are released through budding of the cell's surface. Mainly belonging into this field are viruses causing infectious diseases such as AIDS, hepatitis, hemorrhagic fever, SARS, smallpox, measles, polio and flu. The main object of the invention are agents that contain inhibitors of the protein folding as active ingredients. These include inhibitors of cellular folding enzymes (the enzymatic chaperones) and substances which deregulate the protein folding through chemical chaperones. Due to these agents the highly organised process of assembly and of proteolytical maturation of virus structure proteins is disturbed. As a cause of this, the release and production of infectious progeny viruses is blocked. These agents contain a wide spectrum of efficacy and can therefore be used as new broadly reactive, so called broadband virostatica for the prevention or as a therapy for treatment of diverse virus infections.

The goal of the invention was solved by application of inhibitors of protein folding enzymes. Especially inhibitors of cellular chaperones, such as the heat shock proteins (hsp) have been used. Belonging to this category are agents which hinder the activities of the heat shock proteins Hsp40, Hsp70, Hsp90, Hsp27 and Hsc70, for example the substances Geldanamycin and Deoxyspergualin, which block the activities of the proteins of the Hsp90 and the Hsp/Hsc70 families.

Agents for the treatment of various virus infections were subsequently developed which contained inhibitors that blocked molecular chaperones as active components. Such substances include Geldanamycin, Deoxyspergualin, 4-PBA or Herbimycin A. Substances in form of chemical chaperones are also used that regulate, disturb and block conformation and folding of viral proteins. Such substances include Glycerol, Trimethylamins, Betain, Trehalose or deuterized water (D₂O).

### DETAILED DESCRIPTION OF THE INVENTION

All late processes of virus replication such as assembly, budding, proteolytical maturation and virus release have in common that the virus structure proteins are normally generated as prototype proteins in form of polyproteins. These are then processed into the so called matured virus structure proteins through the activity of proteases which either originate from the host cell, but in most cases represent at least one virally encoded protease. This process is generally known as virus maturation. The highly organised processes of assembly, maturation, budding and release are crucial preconditions for the successful production of progeny viruses. The slightest disturbance of these multi step processes can affect the release and/or the infectivity of progeny virions. All these processes have in common the precise and consecutively attuned processes of protein folding. This means that in the process of assembly, maturation and budding the original conformation of the virus structure proteins, such as they were synthesised by the ribosome, is not maintained, but rather that the secondary and tertiary protein structure of single protein sections and/or the whole virus protein changes a multiple number of times during the process of assembly and maturation.

All methods that disturb the protein folding processes which means the rearrangement of single protein structures will therefore also eliminate the formation of infectious progeny viruses. This can be achieved through interference of folding enzymes which are the cellular or molecular chaperones. Direct influences of the protein folding can be triggered through substances or physical interferences which regulate the protein conformation directly. Generally, so-called chemical chaperones are generally sub-molecular compounds that regulate the amount of structure-bond water on the surface of the protein molecules and hence influence the stability of the secondary structures.

Fields of application are both in the treatment as well as in the prevention of viral infections. Agents for the treatment of various virus infections which contain chemical chaperones in pharmaceutical preparations as effective inhibitors of folding enzymes were subsequently developed. The newly developed drugs developed in accordance with the invention are suitable for the treatment, therapy and inhibition of infections with various human pathogenic and also animal pathogenic viruses. The focus of the invention lies on pathogenic agents causing chronic infectious diseases, such as AIDS (HIV-1 and HIV-2), hepatitis (HCV and HBV), the causative agent of the “Severe Acute Respiratory Syndrome” (SARS), the SRAS-CoV (Corona virus), the smallpox, the causative agents of the viral hemorrhagic fever (VHF), such as the Ebola-viruses as a representative of the Filoviridae family; the causative agents of flu, such as the Influenza-A-Virus.

According to a particular embodiment of the invention various anti-viral effects can be triggered in infected cells. These include for example the induction of apoptosis, which preferentially induces death of cells in the organism. This process is especially caused by the accumulation of immature and in the process of assembly disturbed virus proteins. At the same time the release and the production of infectious progeny virions is blocked due to the inhibition of assembly and the maturation of virus proteins. In the sum of these effects a therapeutic impact can be achieved by blockage of virus replication and the removal of virus producing cells from the infected organism.

The tasks of the invention are solved due to the use of at least one inhibitor of molecular chaperones and/or at least one chemical chaperone. In accordance with the invention pharmaceutical agents were developed for the treatment of virus infections which contain as active ingredients inhibitors of the protein folding in pharmaceutical preparations. In accordance with a preferred degree of execution of the invention agents which hinder, regulate and otherwise influence the activities of molecular chaperones of the host cell are used as inhibitors of the protein folding. These include agents which hinder the activities of the heat shock proteins Hsp40, Hsp70, 90, Hsp27 and Hsc70, for example the substances Geldanamycin, Deoxyspergualin, 4-PBA or Herbimycin A.

A version of the invention consists of the use of substances as chemical chaperones such as Glycerol, Trimethylamine, Betain, Trhalose or deuterized water (D₂O).

In all particular embodiments of the invention these inhibitors and substances are taken up by cells of upper eukaryotes and after cell uptake either block the activities of the molecular chaperones of the host cell, or disturb in form of chemical chaperones the folding of the virus proteins.

In accordance with the invention, substances are used as inhibitors of cellular chaperones or as chemical chaperones. These substances are administered in the various forms in vivo orally, intravenously, intramuscularly, subcutaneously, in form of capsules with or without the occurrence of cell specific changes, or they are administered in other ways. Due to the administration of a specific application and the doses regime those substances show a low degree of zytotoxivity and/or a high selectivity for specific cells and organs, they result in no, or no significant side effects, they exhibit a relatively high metabolite half-life and a relatively low clearance-rate in the organism.

Furthermore, as inhibitors of cellular chaperones or as chemical chaperones whose substances are used in their naturally occurring form isolated from micro organisms and other natural sources, they can be developed through the chemical modification of natural substances, or they can be produced fully synthetically, or they can be synthesized in vivo through gentherapeutical procedures, or they can be produced in vitro or in other micro organisms through genetically engineered procedures.

In accordance with the invention, agents are provided with the inhibitors of cellular chaperones or with the chemical chaperones which surprisingly interfere with the production of infectious progeny and thus block the replication of various viruses and thus the spread of an infection in an organism. Furthermore, they
block the release of infectious viruses from the infected cell,
restrict the expansion of a virus infection in an organism,
interfere with the outbreak of the infectious disease and to the reduction of the spread of infection in the organism (reduction of the “viral load”) of symptom-free virus infected individuals,
prevent the establishment of a systemic virus infection instantaneously after coming in contact with the infectious agent, with infected persons, or being in there closer surroundings,
repress the viral load both during a new infection as well as during a chronic infection, and increase the success of the elimination of a virus infection through one's own immune system and/or through known medicaments which work in combination with the inhibitors of cellular or chemical chaperones in similar or in other ways.

The inhibitors of cellular chaperones or the chemical chaperones can also be used in combination with other anti-virus medicaments and other therapy possibilities, for example with Interferon alpha/beta/gamma and its varieties (for example PEG-modified Interferone), Interleucines, Nucleosidanaloga (Lamivudine, Cidovir, Ribavirin and others), steroids, Thymidikinase-blockers (i.e. Ganzyklovir), plasma exchange, Thymosin alpha 1, vaccines, passive and active vaccination, therapeutical and prophylactical vaccinations, Glycyrrhizin, stem cell transplantation, organ transplantation, nourishment therapy, immunosuppressive, Cyclosporines and derivatives thereof, Amanditin and derivatives thereof, interleukins and other cytokines, non protease-selective protease-inhibitors, Azathioprin, haemodialysis as well as highly active antiretroviral therapy (“HAART”) during a co-infection with HCV or HIV. Since these inhibitors also exhibit an anti-viral effect on HIV, a treatment of HCV/HIV co-infections, especially in combination with HAART-therapy, stands in the center of the application of the invention.

The characteristics of the invention are described in the elements of the claims and the description of the invention, whereas beneficial realizations are presented through both single characteristics as well as several combinations, thereof which protection is requested in this application. The invention also lies in the combined use of known and new elements, the inhibitors of cellular chaperones on the one hand and the chemical chaperones on the other hand. These new pharmaceutical agents which influence the protein folding of virus proteins can furthermore be applied to already acquired anti-viral chemotherapeutics.

In accordance with the invention, the inhibitors of cellular chaperones on the one hand and the chemical chaperones on the other are used in the production of pharmaceutical agents for the control/treatment and prevention of illnesses as well as pathological conditions which
are caused by SARS-CoV and related corona viruses
are triggered through hemorrhagic fever (VHF) in humans and animals, especially in non-human primates (apes) and in animals related to them. Examples for such illnesses infections caused by representatives of the filo viruses, the Ebola-virus and the Marburg-virus or are caused through infections with the Lassa-virus or the Krim/Kongo-hemmorrhagic fever-virus.

For a favourable application of the new kinds of anti-viral agents, according to the invention, for the treatment of virally induced liver diseases (hepatitis), it was noted that in accordance with the invention the use of inhibitors of cellular chaperones or of chemical chaperones consists in the inhibition of the admittance/internalisation and uncoating processes of Flaviviridae as well as the inhibition of the assembly, maturation and release of progeny viruses. The use of the inhibition of the reproduction of Flaviviridae is a result of the following mechanisms
a) blockage/reduction of the assembly and release of new virions,
b) blockage/reduction of the infectivity of released virions,
c) blockage/reduction of the virus expansion in cultivated cells.

This implies that the spread of progeny viruses in infected organs is repressed by the new kind of chaperone inhibitors.

Another use of the inhibitors of cellular chaperones, or of chemical chaperones, lies in the induction of cell death of Hepato-carcinoma cells, further in the oppression and/or prevention of the development of liver cell carcinoma as well as in the therapy of patients with established liver cell carcinomas.

Another use lies in the treatment/therapy/prevention of
HCV-induced liver cirrhosis and/or
HCV-induced liver cell carcinomas
substance induced liver carcinomas
genetically-related liver carcinomas and/or
environmentally related liver carcinomas.

A further use lies in the specifically directed elimination of liver carcinoma cells which developed as a result of
an -HCV-infection and/or
an -HCV-HBV co-infection as well as
an -HCV-HBV-HDV co-infection.

Furthermore inhibitors of cellular chaperones, or chemical chaperones, find their use in the
prevention of the development, the growth and the formation of metastases of liver cell tumors as well as to the preferred destruction of liver carcinoma cells in HCV infected patients,
modulation of the expression, modification and activity of the tumor suppressor-protein p53 and other HCC-relevant tumor suppressor-proteins,
liver cell regeneration in patients with liver inflammation,
reduction of the amount of HCV or HBV infected and virus-producing cells in the liver tissue,
inhibition of both the preservation and persistence of an already established infection as well as of a secondary infection and therefore of the expansion of the infection, including the blockage of the expansion of a HCV-infection in vivo,
treatment of a co-infection with HCV and immune deficiency viruses HIV-1 and HIV-2,
treatment of HCV/HIV-co-infections in combination with the HAART-therapy,
prevention of a re-infection with HCV due to liver or other organ transplantations,
prevention of a re-infection with HCV as a result of cell therapies due to giving the medicament before, during and after a transplantation,
treatment and combat of hepatitis in combination with one another,
prevention of a re-infection with HCV during the transplantation of virus free organs into chronic virus carriers that still have a rest virus and which can infect new organs, as well as during the transfer of virus-infected donor organs into virus-free patients,
prevention of the establishment of a systemic hepatitis virus-infection immediately after coming in contact with the infectious virus or in the,
diminution and elimination of a liver inflammation due to mechanisms of the immune system.

A further use of inhibitors of cellular chaperones or of chemical chaperones lies in the prevention of an establishment of a systemised hepatitis virus-infection immediately after coming in contact with the infectious virus (for example due to pinprick injuries with virus-contaminated blood or blood products).

Another use of inhibitors of cellular chaperones, or of chemical chaperones, is the prevention of a hepatitis virus infection in persons with a high risk of a new infection, for example doctors or other high-risk personnel, drug addicts, travellers to highly endemic regions for hepatitis viruses, in the treatment of patients or for the family members of a chronic virus carrier.

A further usage of inhibitors of cellular chaperones, or of chemical chaperones, lies in the prevention of a re-infection with HCV due to liver or other organ transplantations as well as a result of cell therapies due to giving the medicament before, during and for a while after a transplantation. The administration of the agents is indicated for both the high-risk situation in which virus-free organs are transplanted into chronic virus carriers that always have a rest virus and could infect new organs as well as the transfer of virus-infected donor organs into virus-free patients.

A further usage lies in the treatment of HCV-induced autoimmune illnesses such as the mixed Type II- Cryo-Globulin anaemia.

Another use lies in the combination with medicaments already established in the anti-viral therapy of Hepadna-viruses.

An elementary application of the invention rests in the use of inhibitors of cellular chaperones, or of chemical chaperones, for the production of agents, respectively of pharmaceutical preparations that can be used for the inhibition of the release, maturation and replication of hepatitis viruses as well as for the production of medicaments for the treatment and prophylaxis of an infection with hepatitis viruses.

Another usage is that inhibitors of cellular chaperones, or chemical chaperones, change the post-translative modification of the virus structure proteins and therefore reduce or block the release and infectivity of Flaviviridae.

A further use of inhibitors of cellular chaperones, or of chemical chaperones, lies in the treatment of patients infected with flavi viruses, for example persons that are acutely infected with West-Nil-fever, yellow fever, Dengue-fever (7-day-fever or Dengue hemorrhagic fever) or arbo virus induced encephalitis. Inhibitors of cellular chaperones, or of chemical chaperones, can also be given as a precaution for a virus infection to risk persons such as doctors or travellers to highly endemic regions for the West-Nil-virus, Dengue-fever-virus, yellow fever virus or FSME-virus.

A further example of an application is the treatment of pestivirus-infected farm animals with inhibitors of cellular chaperones, or chemical chaperones.

At the same time, the usage of inhibitors of cellular chaperones, or of chemical chaperones, is innovative in regard to the principal of application. Until now, no known substances/principals/methods have been described which influence the late processes of replication of Hepadna-viruses, especially the release of infectious virions. Also new is that the usage of inhibitors of cellular chaperones, or of chemical chaperones, leads to the blockage of the replication of hepatitis-viruses. In comparison with the so far applied anti-viral methods for the treatment of hepatitis infections whose essential components affect the virus directly, the likely-hood of development of resistance mechanisms during the use of inhibitors of cellular chaperones, or of chemical chaperones, in the treatment of Hepadna-virus infections is magnitudes smaller. The innovative aspect of the effectiveness of inhibitors of cellular chaperones, or of chemical chaperones, makes itself evident in the fact that inhibitors of cellular chaperones, or chemical chaperones, have a wide spectrum of effectiveness towards various hepatitis viruses (HAV, HBV, HCV, HDV, HEV, HGV).

Innovative is also the concept of effectiveness of inhibitors of cellular chaperones, or of chemical chaperones, that may not hinder the virus entry, but therefore impede the production of infectious virus particles of already infected cells with the Hepadna-viruses. They also decrease the release of the virus-encoded E-antigen, which is essential for the establishment of a chronic infection. As a consequence of this activity, the amount of infected virions (virus burden) as well as for an establishment of a chronic infection, the level of E-antigen and thus as a result the virus expansion in vivo is dramatically reduced.

In the sum of these innovative mechanisms, it can be noted that the reduced release of the already few or not even infectious virus particles, in combination with simultaneous cell death of virus-producing carcinoma cells in the case of an in vivo application of inhibitors of cellular chaperones, or of chemical chaperones, results in net effect in the diminish of the amount of infectious virions in an organism infected with Hepadna-viruses. Consequently the total number of infected virus producing cells is reduced. This makes the application of inhibitors of cellular chaperones, or of chemical chaperones, attractive either alone or in combination with therapeutics already established in the anti-viral therapy of Hepadna-viruses.

In another form of the execution of the invention, it was surprisingly found that inhibitors of cellular chaperones, or chemical chaperones, hinder the later processes of the replication cycle of retroviruses. In this case, it was specifically observed that the use, in accordance with the invention, of inhibitors of cellular chaperones, or of chemical chaperones, is suited to interfere with the assembly and release of virions from the cell's surface. During this process, the proteolytic maturation of the virus-structure proteins Gag mediated through the viral protease is blocked. The infectivity of released virions is also reduced. As a result of these innovative activities, inhibitors of cellular chaperones, or chemical chaperones, can subdue virus replication.

The inhibition of the following retroviruses is possible: Spuma-viruses, Mammalian-C-Typ-Onco-viruses, BLV (Bovine Leukemia Virus), HTLV (Human T-Cell Leukaemia Virus), leukaemia viruses, RSV (Rous Sarcoma Virus) or lent viruses. Examples for leukaemia viruses are BLV, HTLV-I or HTLV-II. Examples for lent viruses are Humans Immune Deficiency Virus Type 1 (HIV-1), Humans Immune Deficiency Virus Type 2 (HIV-2), Simian Immune Deficiency Virus (SIV), Feline Immune Deficiency Virus (FIV) or Bovine Immune Deficiency Virus (BIV).

Subject of the invention is also the application of inhibitors of cellular chaperones, or of chemical chaperones, for the therapy/treatment of diseases/pathological conditions which were caused by infections of retroviruses. The diseases/pathological conditions can be caused by infections with leukaemia viruses, human T-cell leukaemia viruses HTLV-I and HTLV-II or by infections with Lenti viruses.

Another area of application of the invention is the combat/treatment of AIDS, both in the early, symptom-free as well as in the advanced stage with the help of inhibitors of cellular chaperones or of chemical chaperones. These substances can also be used in combination with other anti-retroviral medicaments, for example with blockers of the reverse transcriptase and/or the viral protease. The combination with an anti-retroviral therapy based on gentherapeutic interventions is also possible.

Another usage arises due to a combination with the intracellular immunisation such as the insertion of anti-HIV-1/HIV-2 effective genes into stem cells and/or into peripheral CD4⁺T-lymphocytes.

A prevention of a disease outbreak and a reduction in the level of the spread of the infection in the organism (reduction of the “viral load”) of symptom-free HIV-1/HIV-2 seropostive and HIV-1/HIV-2 infected individuals are empirically also possible. Furthermore inhibitors of cellular chaperones, or chemical chaperones, can be used for the treatment/therapy/prevention of HIV-induced demence, especially for the prevention of an HIV-infection of the neurons, glia and endothelial cells in capillaries of the brain. Another use is the interference with the establishment of a systemic HIV-1/HIV-2 infection immediately after coming in contact with the infectious virus (for example due to a pinprick injury with HIV-contaminated blood or blood products).

The principal solution of the invention's goal is shown using HIV-1 and HIV-2 as an example. It is demonstrated that directly after the addition of various substance classes of inhibitors of cellular chaperones, or of chemical chaperones, the production of infectious virus particles is blocked. In accordance with the invention, this phenomenon can be observed both in HIV-1 infected permanent cultures of CD4⁺human T-cells as well as in cultures of human fibroblasts (HeLa-cells) transfected with infectious proviral DNA of HIV-1 and HIV-2. This will be described here in more detail. Due to these new kinds of activities of inhibitors of cellular chaperones, or of chemical chaperones, it can be concluded that the application of in vivo compatible inhibitors of cellular chaperones, or chemical chaperones, can subdue the spread of a future infection in the organism or totally eliminate the virus.

In accordance with the invention, it can be shown that the repressive effect of inhibitors of cellular chaperones, or of chemical chaperones, on the HIV-replication contains the following mechanisms:
1. blockage/reduction of the porteolytical processing of the Gag-polyproteins through the HIV-1 protease;
2. blockage/reduction of the release and budding of new virions on the cell membrane;
3. blockage/reduction of the infectivity of released virions;

4. blockge/reduction of the spread of an infection for HIV-1 in cultured CD4⁺cells.

To find the solution to the task, various protein-chemical, molecular-virological and morphological studies on HIV-1 were conducted within the limits of the invention. In accordance with the invention, the inhibitory affect of cellular chaperones, or chemical chaperones, on the Gag-processing is outlined by means of biochemical methods. To achieve this, HIV-proteins were metabolically pulse labelled with radioactively labelled amino acids, followed by incubation (Chase) in a non-radioactive medium. The information obtained enabled the depiction of the inhibitory effects of inhibitors of cellular chaperones, or of chemical chaperones, on Gag-processing and budding of HIV-virions within short time periods that are adequate to reflect certain stages of the HIV replication cycle.

In accordance to the invention, it is described that the blocking effect of inhibitors of cellular chaperones, or of chemical chaperones, targets the HIV-assembly and release, but not the enzymatic activity of HIV-1 PR. Using in vitro processing studies on isolated Gag- and PR-molecules of HIV-1, it is shown that different substance classes of inhibitors of cellular chaperones, or of chemical chaperones, do not have any influence on the PR-activity.

Also in accordance with the invention, the reduced infectivity of immature HIV-virions as analysed, caused by the effect of inhibitors of cellular chaperones, or of chemical chaperones, is shown by means of end-point-titration studies in CD4⁺T-cells. Hereby, it can be seen that incubation with inhibitors of cellular chaperones, or with chemical chaperones, for less than six hours (adequate to about one-third of the entire HIV replication cycle in the target cell) alone can lead to a ten-fold reduction in the virus-titter and to a fifty-fold reduction of the specific infectivity of the released virus particles.

In accordance with the invention, the influence of inhibitors of cellular chaperones, or of chemical chaperones, onto the morphology of HIV-1-virions in the assembly and budding process along the cell membrane will be analysed. To gain the solution to this task, high-resolution transmission electron microscopy is used for studying HIV-1 infected CD4⁺T-cells. During this, one finds that the treatment with inhibitors of cellular chaperones, or with chemical chaperones, in a time frame of five hours leads to the following changes in the virus morphology:
1. The number of assembling virions arrested in the late stage of the budding phase is significantly increased;

2. the release of virions from the cell surface is disturbed and virus-membrane-conjunctions (“stalk formation”) start to form;
3. the absolute number of virus particles on the cell's surface is reduced;
4. the relative number of immature, cell-free virions is increased.

According with the invention, the inhibitory effect of inhibitors of cellular chaperones, or of chemical chaperones, onto the virus replication in cultures of HIV-1 infected CD4⁺T-cells is demonstrated. The treatment of cells with nanM-concentrations to various substance classes of inhibitors of cellular chaperones or of chemical chaperones interferes with the spread of the infection and brings about the absence of a productive virus replication.

The principle displayed in the description of the invention of the use of inhibitors of cellular chaperones, or of chemical chaperones, for the blockage of an HIV-infection is innovative in regard to the use of an already known substance class (the inhibitors of cellular chaperones, or chemical chaperones) for a new activity (the blockage of Gag-processing and the release of retroviruses).

Furthermore, it is new that the application of inhibitors of cellular chaperones, or of chemical chaperones, for the blockage of HIV and other retroviruses does not target the virus itself, but rather mechanisms that are common for all host cells of the virus. In comparison to previous anti-retroviral methods that effect essential components of the virus itself, the possibility of a development of drug resistances during the use of inhibitors of cellular chaperones, or of chemical chaperones, is by far lesser. The innovation of this principle of inhibitors of cellular chaperones, or chemical chaperones, also shows that these inhibitors have a wide spectrum of efficacy toward various isolates of HIV-1 and HIV-2. The inhibitory effect was observed within the description of the invention with the same intensity of various primary as well as cell-culture-adapted T-cell-troph and makrophage-troph HIV-isolates.

Innovative is also the effect by which inhibitors of cellular chaperones, or chemical chaperones, do not block the entrance of the virus, but disrupt the production of infectious virus particles of already infected cells. Due to this, the amount of infectious virions (virus burden) and therefore the spread of the infection in vivo should be considerably reduced. The average life span of an acutely HIV-infected T-cell adds up to a few days. It is also known that the inhibition of the virus release and due to this the accumulation of at least partially toxic HIV-proteins (especially the envelope-proteins) leads to an increased cytopathic effect and through this to a faster decay of the infected cells. In addition to the inhibition of a new infection, the inhibitory effect of inhibitors of cellular chaperones, or of chemical chaperones, should also lead to a faster death of already infected cells.

In the sum of these innovative mechanisms, it can be noted that in the netto-effect in the case of an in vivo application of inhibitors of cellular chaperones, or of chemical chaperones, the reduced release of the already less- or non-infectious virus particles results in the diminish of the amount of infectious virions in peripherical blood and at the same time of the number of infected producer cells of HIV in the whole organism in combination with simultaneous cell death of virus-producing cells. This makes the usage of inhibitors of cellular chaperones, or of chemical chaperones, attractive by themselves or in combination with already approved enzyme inhibitors used in anti-retroviral therapy.

The principle solution to the invention's goal is shown using the HIV-viruses as examples. In control tests it was initially demonstrated that pre-treatment before hand of target-cells (CD4⁺T-cells and HeLA-cells) with non-zytotoxic concentrations of various substance classes of inhibitors of cellular chaperones, or of chemical chaperones, has no influence regarding the viability of the host cell.

For the solution of the task, molecular-virological, biochemical, immunobiological and electron-microscopic studies on infected cells were conducted in the range of the invention. These cells were infected with various viruses or transfected with viral RNA- and/or DNA-molecules. According with the invention, defects caused by inhibitors of cellular chaperones, or by chemical chaperones, could be determined through the following instruments and methods: (i) virus preparation and determination of infectious titters; (ii) virus-endpoint-titration through microscopic detection of infectious viral particles through plaque-formation and immune histological methods; (iii) cDNA-constructs used through in vitro transcription; (iv) Rnase protection assay for the detection/quantification of viral RNA-molecules; (v) immunofloresence-tests for the determination of the replication capacity of viral RNA-molecules and for the determination of the expansion of the infection; (vi) electron microscopy methods for the analysis of the morphology of viral particles during and after the process of infection; (vii) radioactive pulse-chase- labelling-methods/in vitro translation method for the synthesis of virus structure proteins in vivo and in vitro; (viii) studies on viral proteins by Western blot and immunopricipitation.

The invention shall be illustrated more closely by means of exemplary embodiments, without being limited to these examples.

### EXAMPLE 1

The treatment of Flaviviridae-infected cell cultures with moderate concentrations of inhibitors of cellular chaperones, or of chemical chaperones, drastically reduces the release and spread infectious progeny viruses.

### EXAMPLE 2

The treatment of Flaviviridae-infected cells with inhibitors of cellular chaperones, or with chemical chaperones, leads to differences in the number of in infected cells, detectable virus particles, to changes of the proportion of complete to non-complete virions, as well as to changes in the morphology of secreted progeny viruses.

### EXAMPLE 3

Inhibitors of cellular chaperones, or chemical chaperones, have inhibiting processes and modification of the structure proteins of BVDV and HCV.

### EXAMPLE 4

The treatment of HIV-1 infected cells with inhibitors of cellular chaperones, or with chemical chaperones, reduces the infectivity of released virus particles.

### EXAMPLE 5

Electron microscopic analysis of HIV-1 infected MT-4-cells after the treatment with inhibitors of cellular chaperones, or with chemical chaperones.

### EXAMPLE 6

Inhibitors of cellular chaperones, or chemical chaperones, interfere with the Gag-processing and release of virus from infected T-cell cultures and transfected HeLa-cells.

### EXAMPLE 7

Inhibitors of cellular chaperones, or chemical chaperones interfere with HIV-1 replication in cell cultures.

### EXAMPLE 8

Inhibition of the replication of SARS-CoV in Vero-cells through inhibitors of cellular chaperones, or chemical chaperones.

Literature:

Diamant S., Eliahu N, Rosenthal D, Goloubinoff P. 2001. Chemical Chaperones Regulate Molecular Chaperones in Vitro and in Cells under Combined Salt and Heat Stresses. J. Biol. Chem. 276 (43): 39586-39591.

Gekko K, Timasheff S. 1981. Mechanism of Protein Stabilization by Glycerol: Preferential Hydration in Glycerol-Water Mixtures. Biochemistry. 20:4667-4676.

Kräusslich, H-G; Welker, R (1996). Intracellular transport of retroviral capsid components. Curr. Top. Microbiol. Immunol. 214:25-63.

Nadler S., Tepper M., Schacter B., Mazzucco C. 1992. Interaction of the Immunosuppressant Deoxyspergulaine with a Member of the Hsp70 Family of Heat Shock Proteins. Science 258: 484-486.

Perlmutter D. 2002. Chemical Chaperones: A Pharmacological Strategy for Disorders of Protein Folding and Trafficking. Pediatric Research. 52 (6) 832-836.

Schneider et al. 1996, PNAS 93:14536-14541.

Whitesell et al. 1994, PNAS 91:8324:832.

1. A pharmaceutical preparation comprising an agent for inhibition of assembly and maturation of virus structure proteins, comprising, as an active ingredient, at least one inhibitor of cellular chaperones, or a chemical chaperone.

2. Pharmaceutical preparation according to claim 1, wherein the inhibitors of cellular chaperones, or chemical chaperones comprise substances which hinder, regulate, or otherwise influence folding and proteolytical maturation of virus proteins and through this hinder release and replication of viruses.

3. Pharmaceutical preparation according to claim 1 or 2, wherein the inhibitors of cellular chaperones, or chemical chaperones comprise substances which influence the enzymatic activities of molecular folding enzymes in a host cell.

4. Pharmaceutical preparation according to claim 3, wherein the inhibitors of cellular chaperones, or chemical chaperones comprise substances which are absorbed by cells of higher eucaryotes and after cell absorption block protein convolution of virus structure proteins.

5. Pharmaceutical preparation according to claim 1 or 2, further comprising at least one chemotherapeutic substance having anti-viral effect.

6. Pharmaceutical preparation according to claim 1 or 2, wherein the pharmaceutical preparation is in form for administration in vivo orally, intravenously, intramuscularly, or in encapsulated form with or without cell type specificity determining changes, based on the use of a specific application- and/or doses-regime, shows a low zytotoxivity, triggers no or irrelevant side effects, and shows a relatively high metabolic half-life and a relatively low clearance-rate in an organism to which it is administered.

7. Pharmaceutical preparation according to claim 1 or 2, wherein the inhibitors of cellular chaperones, or chemical chaperones comprise substances which
a) are isolated in their natural form micro-organisms or other natural sources,
b) arise through chemical modifications of natural substances, or
c) are produced totally synthetically, or
d) are synthesised in vivo through gentherapeutical methods.

8. Pharmaceutical preparation according to claim 1 or 2, wherein the inhibitors of cellular chaperones, or chemical chaperones comprise substances which disturb highly organized processes of assembly and of proteolytical maturation of virus structure proteins and through this prevent the release and production of descendant viruses.

9. Pharmaceutical preparation according to claim 1 or 2, wherein the inhibitors of cellular chaperones, or chemical chaperones comprise substances which regulate, disturb or block the folding of viral proteins.

10. Pharmaceutical preparation according to claim 1 or 2, wherein the inhibitors of cellular chaperones, or chemical chaperones comprise substances which disturb the later processes of virus replication comprising assembly, budding, proteolytical maturation and virus release.

11. Pharmaceutical preparation according to claim 1 or 2, wherein the inhibitors of cellular chaperones, or chemical chaperones comprise substances which disturb the proteolytic maturation of precursor proteins of viral polyproteins.

12. Pharmaceutical preparation according to claim 1 or 2, wherein the inhibitors of cellular chaperones, or chemical chaperones comprise substances which block the activity of viral proteases.

13. Pharmaceutical preparation according to claim 1 or 2, wherein the inhibitors of cellular chaperones, or chemical chaperones comprise substances which disturb activities of cellular proteases that are involved in virus maturation.

14. Pharmaceutical preparation according to claim 1 or 2, wherein the inhibitors of cellular chaperones, or chemical chaperones comprise substances which exhibit a wide spectrum of efficacy as broadband virostatica for prophylaxis and/or for therapy of virus infections.

15. Pharmaceutical preparation according to claim 1 or 2, wherein the inhibitors of cellular chaperones, or chemical chaperones comprise substances which hinder activities of cellular chaperones.

16. Pharmaceutical preparation according to claim 13, wherein the inhibitors of cellular chaperones, or chemical chaperones comprise substances which hinder the activities of the heat shock proteins Hsp40, Hsp70, 90, Hso27 and Hsc70.

17. Pharmaceutical preparation according to claim 1 or 2, wherein the inhibitors of cellular chaperones or chemical chaperones comprise: Geldanamycin, Deoxyspergualin, 4-PBA or Herbimycin A.

18. Pharmaceutical preparation according to claim 1 or 2, wherein the chemical chaperones comprise substances which regulate, disturb or block protein conformation and folding of viral proteins.

19. Pharmaceutical preparation according to claim 18, wherein the chemical chaperones comprise Glycerol, Trimethylamines, Betain, Trehalose or deuterized water (D₂O).

20. Pharmaceutical preparation according to claim 18, wherein the chemical chaperones comprise substances for the treatment, therapy and inhibition of infections with various human or animal pathogenic viruses.

21. Pharmaceutical preparation according to claim 1 or 2, wherein the inhibitors of cellular chaperones, or chemical chaperones comprise substances for the treatment, therapy and inhibition of infections with causative pathogens of chronically infectious diseases comprising AIDS (HIV-1 and HIV-2), hepatitis (HCV and HBV), “Severe Acute Respiratory Symptoms” (SARS), SARS-CoV (Corona virus), smallpox, viral hemorrhagic fever (VHF) or influenza.

22. Method for inhibition of assembly and maturation of virus structure proteins in an organism. comprising administering a pharmaceutical preparation of claim 1 to the organism.

23. Method for inhibition of entry/internalization process, replication, and maturation and release of Flaviviridae in an organism, comprising administering a pharmaceutical preparation of claim 1 to the organism.

24. Method for inhibition of processes in the life cycle of Flaviviridae after maturation and release thereof in an organism, comprising administering a pharmaceutical preparation of claim 1 to the organism.

25. Method according to claim 22 wherein the inhibitors of cellular chaperones, or chemical chaperones, at least substantially, through blockage, prevent production of infectious virions of Flaviviridae-infected cells.

26. Method according to claim 22, wherein the inhibitors of cellular chaperones, or chemical chaperones cause inhibition of release of virions as well as substantially complete reduction of infectivity of released virions.

27. Method according to claim 22, wherein the inhibitors of cellular chaperones, or chemical chaperones repress virus reproduction and through this a new infection of host cells and therefore spread of an infection in vivo, the infection being hepatitis-C-virus in liver tissue of an infected person.

28. Method according to claim 22 for inhibition of reproduction of Flaviviridae wherein the inhibitors of cellular chaperones, or chemical chaperones effect
a) blockage/reduction of release of new virions,
b) blockage/reduction of infectivity of released virions,
c) blockage/reduction of infection expansion in cultures of a host cell, or
lockage/reduction of infection expansion in infected organs in vivo.

29. Method according to claim 22, wherein the organism is a human or animal infected with a Flavivirus or a Pestivirus and the pharmaceutical preparation represses said infection.

30. Method according to claim 22, wherein the organism is a human or animal having Hepato-carcinoma cells and the pharmaceutical preparation induces death of said cells.

31. Method according to claim 22 wherein the organism is a human or an animal and the pharmaceutical preparation represses and/or prevents development of liver cell carcinomas in the human or the animal.

32. Method according to claim 30 or 31, wherein the human or the animal has established liver cell carcinomas.

33. Method for treating or preventing in a human being
HCV-induced liver cirrhosis, or
HCV-induced liver cell carcinomas, or
medicine-induced liver carcinomas, or
genetically conditioned liver carcinomas, or
through the environment induced liver carcinomas, or
through a combination of viral and non-viral factors induced liver carcinomas,
comprising administering to the human a pharmaceutical preparation according to claim 1.

34. Method for elimination of liver carcinoma cells in a human which develop as the result of an
HCV-infection, or
corresponding co-infection of HCV and HBV, or
HDV/HBV/HCV co-infection, or
HIV/HCV co-infection, or
HCV and a co-infection with other viruses, bacteria or parasites, comprising administering to the human a pharmaceutical preparation according to claim 1.

35. Method according to claim 29, wherein the organism is a human who has been infected with a Flavivirus.

36. Method according to claim 29, wherein the organism is a farm animal which has been infected with a Falvivirus or a Pestivirus.

37. Method for reduction of number of infected virus-producing cells in liver cell tissue of a human or an animal, comprising administering a pharmaceutical preparation of claim 1 to the human or the animal.

38. Method for prophylactic treatment of a human who may be exposed to a Flavivirus, comprising administering a pharmaceutical preparation of claim 1 to the human, wherein the inhibitors of cellular chaperones, or chemical chaperones change post-translational modification and proteolytic processing of Flaviviridae structure proteins as well as reduce dimerization of virus-envelope-proteins and through this reduce or block release and infectivity of Flaviviridae.

39. Method according to claim 38, wherein infection of the human or the animal with the virus is already established and the pharmaceutical preparation inhibits the infection, as well as secondary infection, and therefore for spread of infection, the inhibiting comprising blockage of expansion of a Flaviviridae-infection in vivo.

40. Method for treatment of HCV caused hepatitis, Flavivirus caused fever, haemorrhages and encephalitis and Pestivirus caused illnesses in a human, comprising administering a pharmaceutical preparation of claim 1 to the human.

41. Method according to claim 40, wherein the human is infected with a Flavivirus and the method further comprises administering to the human at least one other anti-viral pharmaceutical for Flaviviridae-infections.

42. Method according to claim 40, or 41, wherein the human has co-infections of Flaviviruses and Pestiviruses and the administration of the pharmaceutical preparation treats the co-infections.

43. Method for treatment of humans having co-infections of HCV and immune deficiency viruses HIV-1 and HIV-2, comprising administering a pharmaceutical preparation of claim 1 to the human.

44. Method for treatment of HCV/HIV-co-infections in a human comprising administering to the human HAART-therapy and a pharmaceutical preparation of claim 1.

45. Method for prevention of a re-infection with HCV during liver and other organ transplantations in a human patient, comprising administering a pharmaceutical preparation of claim 1 to the human patent.

46. Method for prevention of a re-infection with HCV during cell therapies of a liver or other organ transplantation in a human patient, comprising administering a pharmaceutical preparation of claim 1 to the client before, during and after the transplantation.

47. Method according to claim 45 or 46 for the prevention of a re-infection with HCV during the transplantation of the organ, wherein the organ is virus-free and the patient is a chronic virus carrier or the organ is virus-infected and the patient is virus-free.

48. Method according to claim 38, wherein the human comprises doctors and other health workers, residents of residences frequently visited by many other people, drug addicts, travellers in highly endemic regions for Flaviviridae, and relatives of chronic virus carriers.

49. (canceled)

50. Method for the inhibition of an already established infection as well as a secondary infection and therefore the spread of the infection in an organism, including the blockage of the expansion of an HBC-infection in vivo, comprising administering a pharmaceutical preparation of claim 1 to the organism.

51. Method for treatment of hepatitis in a human, comprising administering a pharmaceutical preparation of claim 1 to the human.

52. Method according to claim 51, further comprising also administering to the human another pharmaceutical for anti-viral therapy of Hepadna-viruses.

53. Method for treatment of a co-infection with HBV and immune deficiency viruses HIV-1 and HIV-2 in a human, comprising administering a pharmaceutical preparation of claim 1 to the patient.

54. Method for treatment of HBV/HIV-co-infections in a human, comprising administering to the human a pharmaceutical preparation of claim 1 and HAART-therapy.

55. Method for inhibition of the release, maturation and replication of hepatitis viruses in a human, comprising administering a pharmaceutical preparation of claim 1 to the human.

56. Method for treatment and prophylaxis of hepatitis in a human, comprising administering a pharmaceutical preparation of claim 1 to the human.

57. Method for treatment of infections due to hepatitis or retro viruses in a human, comprising administering a pharmaceutical preparation of claim 1 to the human.

58. Method for inhibition of release, maturation and replication of retro viruses in a human, comprising administering to the human a pharmaceutical preparation of claim 1.

59. Method for inhibition of late stages of replication cycle of retro viruses and hepatitis viruses in a human, comprising administering to the human a pharmaceutical preparation of claim 1.

60. Method for hindering assembly and release of virions from surfaces of cells in an organism, comprising administering a pharmaceutical preparation of claim 1 to the organism.

61. Method of hindering, in retroviruses in an organism, proteolytic processing of structural Gag proteins through viral protease, comprising administering a pharmaceutical preparation of claim 1 to the organism.

62. Method of inhibiting in an organism release, maturation and replication of
a) Spuma-viruses, or
b) Mammalian C-Type Onco-viruses, or
c) BLV (Bovine Leukemia Virus), or
d) HTLV (Human T-Cell Leukemia Virus), or
e) Leukaemia viruses, or
f) RSV (Rous Sarcoma Virus) viruses, or
g) Lenti-viruses,
comprising administering a pharmaceutical preparation of claim 1 to the organism.

63. Method according to claim 62, wherein the release, maturation and replication of
a) HTLV-I or
b) HTVL-II
is hindered.

64. Method according to claim 62, wherein the viruses of which the release, maturation and replication is hindered are Lenti viruses which are
a) Humans Immune Deficiency Virus Type 1 (HIV-1), or
b) Humans Immune Deficiency Virus Type 2 (HIV-2), or
c) Apes Immune Deficiency Virus (SIV), or
d) Cats Immune Deficiency Virus (FIV), or
e) Cattle Immune Deficiency Virus (BIV)

65. (canceled)

66. Method for treatment of AIDS in a human, comprising administering a pharmaceutical preparation of claim 1 to the human.

67. Method according to claim 66, further comprising administering to the human at least one other anti-retroviral agent, blocker of reverse trascriptive and/or of protease of the virus, anti-viral therapy based on gentherapeutical intervention, intracellular immunization or introduction of anti-HIV-1/HIV-2 effective genes into stem cells and /or peripheral CD4+ lymphocytes.

68. Method for treatment of AIDS in an advanced state of disease in a human, comprising administering a pharmaceutical preparation of claim 1 to the human.

69. Method for prevention of an outbreak of HIV-1/HIV-2 infection in humans and for reduction of spread of infection in symptom-free HIV-1/HIV-2 seropositive and HIV-1/HIV-2 infected humans, comprising administering a pharmaceutical preparation of claim 1 to the humans.

70. Method for treatment and prevention of HIV-induced dementia in a human by prevention of HIV-infection of neurons, Glia-, and Endothel-cells in the capillaries of the brain of the human, comprising administering a pharmaceutical preparation of claim 1 to the human.

71. Method for prevention of establishment of a systemic HIV-1/HIV-2-infection in a human directly after the human has come in contact with infectious HIV-1/HIV-2 viruses, comprising administering a pharmaceutical preparation of claim 1 to the human.

72. Pharmaceutical preparation according to claim 2, wherein the viruses said release and replication of which is hindered by said inhibitors of cellular chaperones or chemical chaperones comprise viruses for AIDS, hepatitis, hemorrhagic fever, SARS, smallpox, measles, polio, herpes or influenza. 6cm 73. Pharmaceutical preparation according to claim 15, wherein the substances which block cellular comprise heat shock proteins.
